(19) United States
(12) Patent Application Publication
(10) Pub. No.: US 2016/0184016 A1
(43) Pub. Date: Jun. 30, 2016

(54) System and Method of Managing Portable Laser Therapy Apparatus

(71) Applicant: K-LASER, LLC, Franklin, TN (US)

(72) Inventor: Richard Albright, Franklin, TN (US)

(21) Appl. No.: 14/798,121

(22) Filed: Jul. 13, 2015

Related U.S. Application Data
(63) Continuation-in-part of application No. 13/470,976, filed on May 14, 2012, now abandoned.

(51) Int. Cl. A61B 18/20; G06Q 20/18; G06Q 20/06; A61N 5/06; G06Q 50/22

(52) U.S. Cl. CPC A61B 18/203; A61N 5/0616; G06Q 50/22; G06Q 20/06; G06Q 20/18; A61N 2005/0644; A61N 2005/067

(57) ABSTRACT

Provided is a method of using a computer system to manage a remote laser emitting apparatus used in medical treatments, including the computer system receiving, through computer data communication, apparatus identification information associated with a remotely located laser emitting apparatus, and user identification information associated with a user of the laser emitting apparatus, the computer system storing the apparatus identification information and user identification information, the computer system correlating, in response to receiving data regarding a purchase of one or more treatment credits by the user, the purchased treatment credits to the laser emitting apparatus associated with the user, and the computer system communicating the purchased treatment credits to the laser emitting apparatus through computer data communication, wherein the purchased treatment credits authorize the laser emitting apparatus to perform a number of treatments corresponding to the purchased treatment credits.

### CROSS-REFERENCE TO RELATED APPLICATIONS

This application is a Continuation-In-Part of U.S. application Ser. No. 13/470,976, filed on May 14, 2012.

### FIELD OF INVENTION

The present general inventive concept relates generally to a portable apparatus to perform laser therapy, and, more particularly, a portable apparatus to apply lasers of different wavelengths and powers to living tissue for therapeutic treatment and/or surgery.

### BACKGROUND

Laser light therapy has become increasingly popular in physiotherapy and surgery applications due to the many benefits available through the application of laser light. Laser light can be used to treat a variety of problems, ranging from relatively mild conditions, such as acne and skin wrinkling, to more complex problems lying deep under the skin, including afflictions of both organs and bones. In many cases, the application of laser therapy may negate the need for conventional pharmaceutical and/or surgical procedures. Different powers, wavelengths, and frequencies are used to target the distinct tissue types associated with the different medical conditions being treated. With the many physical benefits available over the large range of these powers, wavelengths, and frequencies, there exists a need for a device to deliver a large number of different combinations of these values in order to treat a wide variety of conditions. Further, in order for the device to be readily adapted in home and field use as well as medical office and clinical conditions, the device should be readily portable, updatable, and relatively easy to use.

### BRIEF SUMMARY

The present general inventive concept provides a readily portable laser emitting apparatus to conveniently apply laser emissions to a patient for therapeutic treatment and/or surgery. The portable laser emitting apparatus includes one or more laser sources, and may be controlled so as to apply a variety of levels of laser emissions according to different desired therapeutic and surgical procedures.

Additional aspects and advantages of the present general inventive concept will be set forth in part in the description which follows, and, in part, will be obvious from the description, or may be learned by practice of the present general inventive concept.

The foregoing and/or other aspects and advantages of the present general inventive concept may be achieved by a portable laser emitting apparatus to be used in physiotherapy and/or surgery, the apparatus including a readily portable housing, one or more laser sources of same or different wavelengths provided in the housing, and a flexible waveguide extending from the housing to transmit laser light from the one or more laser sources to a target area.

The flexible waveguide may include an optical fiber with a core size of approximately 200 um, and an NA of approximately 0.15 to 0.37.

The apparatus may further include a handpiece, provided at a distal end of the flexible waveguide, configured to emit the laser light to the target area.

The handpiece may be configured to be selectively controlled to deliver the laser light in a focus mode or a zoom mode.

The apparatus may further include at least two detachable members to be selectively attached to the handpiece according to selection of the focus mode or the zoom mode.

At least one of the detachable members may be a twist control that is twisted to adjust a contact area of the transmitted laser light in the zoom mode.

A spot size of the laser light delivered in the zoom mode may be adjustable from approximately 1 to 5 cm².

The detachable members may be mechanically or magnetically coupled to the handpiece.

The apparatus may further include a combiner to combine light from the one or more laser sources into the laser light transmitted by the flexible waveguide.

The flexible waveguide may be provided with a pliable metal sheath surrounding the flexible waveguide and control wiring connected to the handpiece.

The one or more laser sources may include a first laser source to transmit laser light having a wavelength of approximately 660 nm at a power up to approximately 100 mW, and a second laser source to transmit laser light having a wavelength of approximately 800 nm in a power range of approximately 0.1 to 12.0 W.

The one or more laser sources may further include a third laser source to transmit laser light having a wavelength of approximately 970 nm at a power range of approximately 0.1 to 12.0 W.

The second and/or third laser source may transmit at a power range of approximately 0.1 to 8.0 W.

The one or more laser sources may further include a fourth laser source to transmit laser light having a wavelength of approximately 905 nm at a power range of approximately 0.1 to 12.0 W.

The frequencies of the laser light may be adjustable between approximately 1 to 20,000 Hz in approximately 1 Hz increments.

Any of the one or more laser sources may be controlled to emit the laser light separately or concurrently.

The apparatus may further include a carrying handle provided to the housing so that the apparatus may be transported by hand by a user.

The apparatus may further include a data storage to store a plurality of predetermined settings of wavelength and power combinations to be emitted from the one or more laser sources, and a controller to control the one or more laser sources to operate according to the predetermined settings.

The apparatus may further include a communication terminal to receive data updates for the controller and/or data storage.

The communication terminal may be a USB port.

The communication terminal may perform wireless communication.

The apparatus may further include a touch screen user interface.

The apparatus may further include a rechargeable battery to supply power to the apparatus.

The one or more laser sources may be light emitting diodes.

The foregoing and/or other aspects and advantages of the present general inventive concept may also be achieved by a method of using a computer system to manage a remote laser emitting apparatus used in medical treatments, including the computer system receiving, through computer data communication, apparatus identification information associated with a remotely located laser emitting apparatus, and user identification information associated with a user of the laser emitting apparatus, the computer system storing the apparatus identification information and user identification information, the computer system correlating, in response to receiving data regarding a purchase of one or more treatment credits by the user, the purchased treatment credits to the laser emitting apparatus associated with the user, and the computer system communicating the purchased treatment credits to the laser emitting apparatus through computer data communication, wherein the purchased treatment credits authorize the laser emitting apparatus to perform a number of treatments corresponding to the purchased treatment credits.

The foregoing and/or other aspects and advantages of the present general inventive concept may also be achieved by a portable laser emitting apparatus to be used in physiotherapy and/or surgery, the apparatus including a readily portable housing, one or more laser sources of same or different wavelengths provided in the housing, a flexible waveguide extending from the housing and configured to transmit laser light from the one or more laser sources to a target area, a data storage configured to store a plurality of predetermined settings of wavelength and power combinations to be emitted from the one or more laser sources, data corresponding to past usage of the laser emitting apparatus, and treatment credit information regarding whether the laser emitting apparatus is authorized to perform one or more treatments, a communication terminal configured to receive data updates for the controller and/or data storage from a remote device management system, and a controller configured to control the one or more laser sources to operate according to the predetermined settings in response to the laser emitting apparatus being authorized to perform a treatment, the past usage data, and communication with the remote device management system to share the usage data and/or receive new treatment credits.

The foregoing and/or other aspects and advantages of the present general inventive concept may also be achieved by a system to perform laser therapy, the system including a portable laser emitting apparatus configured to perform laser therapy, and a remotely located computer system configured to receive, through computer data communication, apparatus identification information associated with the laser emitting apparatus, and user identification information associated with a user of the laser emitting apparatus, to store the apparatus identification information and user identification information, to correlate, in response to receiving data regarding a purchase of one or more treatment credits by the user, the purchased treatment credits to the laser emitting apparatus associated with the user, and to communicate the purchased treatment credits to the laser emitting apparatus through computer data communication, wherein the purchased treatment credits authorize the laser emitting apparatus to perform a number of treatments corresponding to the purchased treatment credits.

Other features and aspects may be apparent from the following detailed description, the drawings, and the claims.

### BRIEF DESCRIPTION OF THE FIGURES

The following example embodiments are representative of example techniques and structures designed to carry out the objects of the present general inventive concept, but the present general inventive concept is not limited to these example embodiments. In the accompanying drawings and illustrations, the sizes and relative sizes, shapes, and qualities of lines, entities, and regions may be exaggerated for clarity. A wide variety of additional embodiments will be more readily understood and appreciated through the following detailed description of the example embodiments, with reference to the accompanying drawings in which:

FIG. 1 illustrates a portable laser emitting apparatus according to an example embodiment of the present general inventive concept;

FIG. 2 is a schematic illustration of some of the components of the portable laser emitting apparatus of FIG. 1, according to an example embodiment of the present general inventive concept;

FIG. 3 illustrates example elements and functions of the handpiece of the portable laser emitting apparatus according to an example of the present general inventive concept;

FIG. 4 is a graph illustrating the application of the laser light in the continuous wave mode;

FIG. 5 is a graph illustrating the application of the laser light in the frequency modulated, or pulsed emission, mode;

FIG. 6 is a graph illustrating the application of the laser light in the intense super pulse mode;

FIG. 7 illustrates an available treatments display used to track and control operations of the portable laser emitting apparatus according to an example embodiment of the present general inventive concept;

FIG. 8 illustrates a purchase options display used to allow a user to choose from a plurality of purchase options according to an example embodiment of the present general inventive concept;

FIG. 9 illustrates an expected revenue display to track and control operations of the portable laser emitting apparatus according to an example embodiment of the present general inventive concept;

FIG. 10 illustrates a history display used to track and control operations of the portable laser emitting apparatus according to an example embodiment of the present general inventive concept;

FIG. 11 illustrates number of treatments information that may be displayed and associated with the history display of FIG. 9;

FIG. 12 illustrates anatomies treated information that may be displayed and associated with the history display of FIG. 9;

FIG. 13 illustrates total number of treatments information that may be displayed and associated with the history display of FIG. 9;

FIG. 14 illustrates treatments purchased and credit units information that may be displayed and associated with the history display of FIG. 9;

FIG. 15 illustrates a dashboard display including the displays illustrated in FIGS. 7 and 9-14 according to an example embodiment of the present general inventive concept;

FIG. 16 illustrates a logged in display through which a user may update a user profile according to an example embodiment of the present general inventive concept;

FIG. 17 illustrates a user profile display according to an example embodiment of the present general inventive concept;

FIG. 18 illustrates an updatable information display for the user to edit user information according to an example embodiment of the present general inventive concept;

FIG. 19 illustrates a successful update notification display according to an example embodiment of the present general inventive concept;

FIG. 20 illustrates another example of the available treatment display of FIG. 7 according to an example embodiment of the present general inventive concept;

FIGS. 21-23 illustrate example interactive displays used to purchase treatment credits through the laser emitting apparatus according to an example embodiment of the present general inventive concept;

FIG. 24 illustrates a method of controlling the portable laser emitting apparatus according to an example embodiment of the present general inventive concept; and

FIG. 25 illustrates a system to perform laser therapy including a computer system in communication with a portable laser emitting apparatus, laptop computer, and/or smartphone according to an example embodiment of the present general inventive concept.

### DETAILED DESCRIPTION

Reference will now be made to various example embodiments of the present general inventive concept, examples of which are illustrated in the accompanying drawings and illustrations. The example embodiments are described herein in order to explain the present general inventive concept by referring to the figures.

The following detailed description is provided to assist the reader in gaining a comprehensive understanding of the methods, apparatuses, and/or systems described herein. Accordingly, various changes, modifications, and equivalents of the methods, apparatuses, and/or systems described herein will be suggested to those of ordinary skill in the art. The described progression of processing operations described are merely examples, however, and the sequence of operations is not limited to that set forth herein and may be changed as is known in the art, with the exception of operations necessarily occurring in a certain order. Also, description of well-known functions and constructions may be omitted for increased clarity and conciseness.

Various embodiments of the present general inventive concept, as described herein, provide a laser emitting apparatus that is lightweight and readily portable. In other words, the apparatus is designed so as to be easily transported from room to room, or between different locations in the same room, and therefore may be conveniently used in a home or office environment. The portable laser emitting apparatus may be used for physiotherapy and/or surgery on human and/or animal subjects, and may also have application on non-living subjects. Such various applications will be evident to one skilled in the art. In most of the various example embodiments described herein, a target area of a human subject is discussed, and the human subject may be referred to as the patient. However, it is understood that the use of laser light emitted from the portable laser emitting apparatus is not limited to such an application.

To further increase the ease of use of the portable laser emitting apparatus, according to various embodiments of the present general inventive concept, one or more laser sources of same or different wavelengths are provided in a housing of the portable laser emitting apparatus, and the laser light emitted from these laser sources is transmitted through a flexible waveguide to then be applied to a target area of the patient.

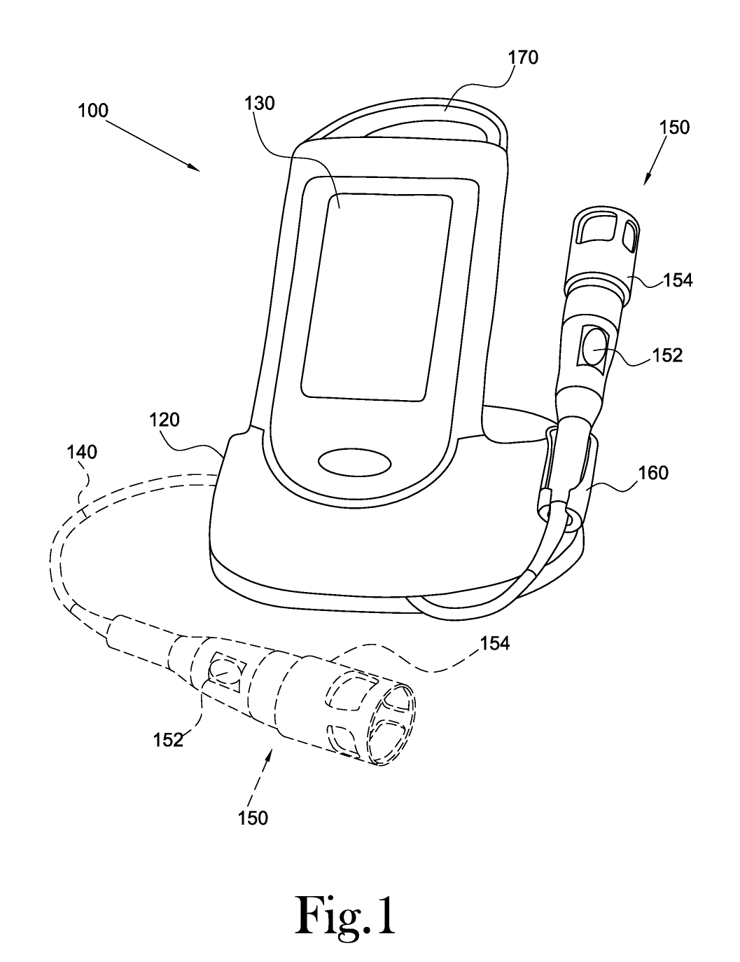

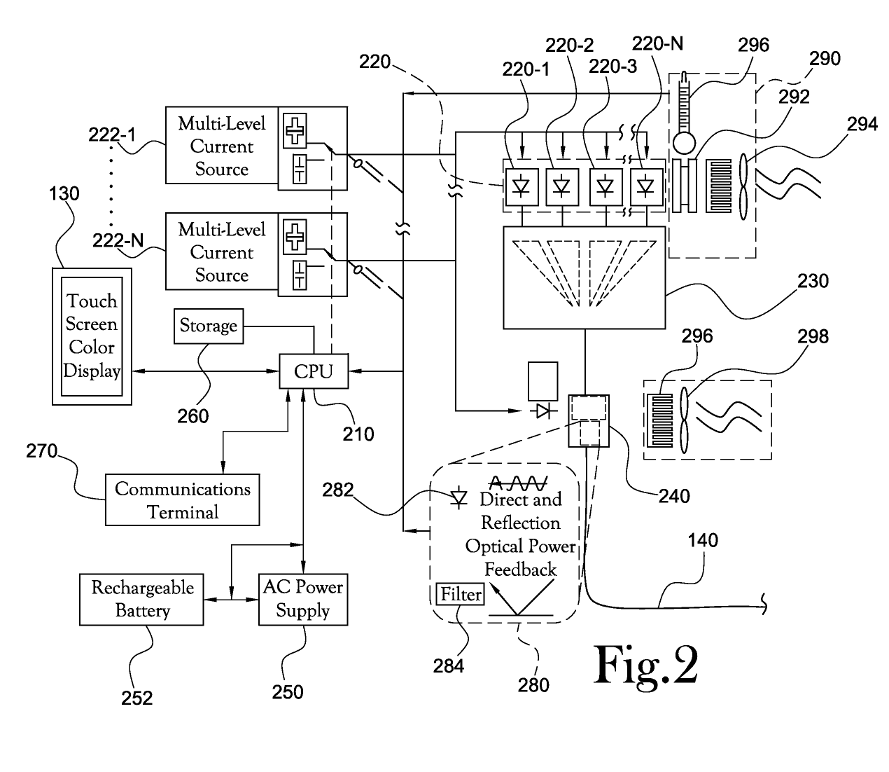

FIG. 1 illustrates a portable laser emitting apparatus according to an example embodiment of the present general inventive concept. Referring to FIG. 1, this example embodiment of the portable laser emitting apparatus 100 includes housing 120 to enclose and protect many of the components of the laser emitting apparatus 100, a touch screen display 130 to provide user interaction with the portable laser emitting apparatus, a flexible waveguide 140 to transmit laser light away from light sources provided in the housing 120 (discussed in more detail in the description of FIG. 2), and a handpiece 150 provided at a distal end of the flexible waveguide 140 to emit the laser light delivered through the flexible waveguide 140 to a target area of a patient. The example embodiment illustrated in FIG. 1 also includes a handpiece docking portion 160 which my secure the handpiece 140 when the portable laser emitting apparatus 100 is not in use, as well as a handle 170 to increase the convenience enjoyed by a user when transporting the device. To further increase the user's convenience regarding portability, in some example embodiments the portable laser emitting apparatus 100 is constructed of such lightweight material(s) as to weigh less than or equal to approximately 3.5 pounds. In other words, the portable laser emitting apparatus 100 is fabricated so as to be readily carried by a user, such as by hand, to easily move from room to room without aid from other people or mechanical aid.

It will be understood by one skilled in the art that various example embodiments of the present general inventive concept may omit various elements described in regard to the illustrated example embodiments, and that various other elements may be added. Further, the configurations of the portable laser emitting apparatus 100 are merely example configurations, and may be altered according to the various design and/or use preferences. Various components, such as the touch screen display 130, may be integrated along with the housing 120 of the portable laser emitting apparatus 100, or may be modular in order to be readily removed in the event that repair or replacement is desired.

Although not illustrated in FIG. 1, the portable laser emitting apparatus 100 may be provided with a power connection, such as an electrical cord, to be connected with an AC power source, as well as a rechargeable battery which may be provided inside or outside of the housing 120. In various example embodiments, the rechargeable battery may be removable from the portable laser emitting apparatus 100 to be recharged, and in other various example embodiments the rechargeable battery may be recharged solely by power supplied by the AC power source. In other various example embodiments of the present general inventive concept, the portable laser emitting apparatus may be equipped to receive power through wireless power transmission.

Although a touch screen display 130 is illustrated in FIG. 1, it will be understood by one skilled in the art that various other user interfaces may be employed in the portable laser emitting apparatus. For example, a conventional display and keypad combination may be substituted for the touch screen display 130. In various example embodiments, the touch screen display 130 can display images in 16 million color combinations.

The flexible waveguide 140 may be an optical cable, such as a single emitting fiber with a core size of approximately 200 um, and an NA of approximately 0.15 to 0.37. However, the waveguide 140 is not limited to an optical cable, nor an optical cable with these example attributes.

The flexible waveguide 140 may be provided with a pliable metal sheath surrounding the flexible waveguide to protect the flexible waveguide 140, and to house control wiring connected to the handpiece 150.

The example handpiece 150 illustrated in FIG. 1 is provided with at least one switch 152 to enable a user to switch between various laser application modes, and/or to start and stop emission of laser light from the handpiece 150, which will be discussed in more detail later in this description. The example handpiece 150 is also provided with a twist control 154 to enable a user to adjust the focus area of the laser application in a zoom mode, which will also be discussed in more detail later in this description. It will be understood that the at least one switch 152 and twist control 154 are merely example embodiments of the controls provided to the handpiece 150 to aid the operation by a user. Other types of controls may be added or substituted on the handpiece, or such controls may be located on the housing 120 of the portable laser emitting apparatus 100. For example, control of the laser application modes and focus areas may be controlled through the touch screen display 130 illustrated in FIG. 1.

FIG. 2 is a schematic illustration of some of the components of the portable laser emitting apparatus 100 of FIG. 1, according to an example embodiment of the present general inventive concept. According to the example embodiment illustrated in FIG. 2, the portable laser emitting apparatus 100 is provided with a CPU 210 to control the functions of the various provided components. The CPU 210 is in electrical communication with the touch screen display 130, such that commands entered by the user are then carried out by the CPU 210, and results associated with the commands, as well as other various feedback, are displayed to the user.

One or more laser sources 220 may be provided inside the housing 120. In the example embodiment illustrated in FIG. 2, multiple laser sources 220-1 through 220-N are provided inside the housing 120. However, it will be understood that any number of laser sources 220 may be provided, including a single laser source 220. The one or more laser sources 220 may have the same or different output wavelengths and/or powers. In various example embodiments, the one or more laser sources 220 may be light emitting diodes, and may be controlled to operate separately or concurrently.

In an example embodiment such as the one illustrated in FIG. 2, in which multiple laser sources 220 are provided in the housing 120, an optical combiner 230 may be provided to combine the outputs of the multiple laser sources 220 before the outputs enter the flexible waveguide 140.

The portable laser emitting apparatus may also be provided with an optical coupler 240 coupled at some point to the flexible waveguide 140. An additional laser source 242 may be provided to the optical coupler 240, and may have a wavelength between approximately 400 nm and 700 nm. The additional laser source 242 may be collimated at the emission point of the optical coupler 240, and aligned with the flexible waveguide 140. In various example embodiments, the additional laser source 242 may emit light to serve as a guiding beam to indicate an approximate point at which the energy is delivered on the target, i.e., on the tissue, and thus may function as a pointing ray, and/or in various example embodiments the additional laser source 242 may function as an additional therapeutic laser source having a biostimulating effect that may be collimated at the emission point of the optical coupler 240 and aligned with the flexible waveguide 140.

In the example embodiment illustrated in FIG. 2, the output of each of the one or more laser sources 220 are respectively controlled by corresponding one or more multi-level current sources 222-1 through 222-N. The multi-level current sources 222-1 through 222-N are controlled by the CPU 210 to adjust the output of the one or more laser sources 220 to the respective desired levels. The multi-level current sources 222-1 through 222-N may maintain the light output of the respective one or more laser sources 220 constant within 1%.

As previously discussed in regard to FIG. 1, power may be provided to the portable laser emitting apparatus 100 through an AC power supply 250, either connected to a source such as a wall adapter or received wirelessly, or through a rechargeable battery 252. According to various example embodiments, the rechargeable battery may be an internally or externally provided LiFePO4 battery, which may be removable for charging, or may be fixed and charged through the AC power supply 250.

In the example embodiment illustrated in FIG. 2, a data storage 260 is provided in electrical communication with the CPU 210 in order to store a number of preset levels to control output of the one or more laser sources 220. According an example embodiment, a total of 55 different output levels may be stored in the data storage 260, and may be easily selected by the user rather than fine-tuning the outputs of the one or more laser sources 220 to desired levels. The data storage 260 may also be used to store a control system used by the CPU 210 to control the operations of the portable laser emitting apparatus 100. Any of a number of data storage devices, such as, for example, a flash memory device, may be provided as the data storage 260.

A communication terminal 270 is also provided to the example embodiment illustrated in FIG. 2. The communication terminal may be used to provide any system and/or memory updates to the portable laser emitting apparatus 100. For example, the control system or output level presets may be updated or expanded. The communication terminal may be, for example, a USB port used to connect the portable laser emitting apparatus 100 to a computer or other electronic device to download the update data. As another example, the communication terminal may be a wireless connector, such as a Wi-Fi connector, to receive the update data in a wireless fashion.

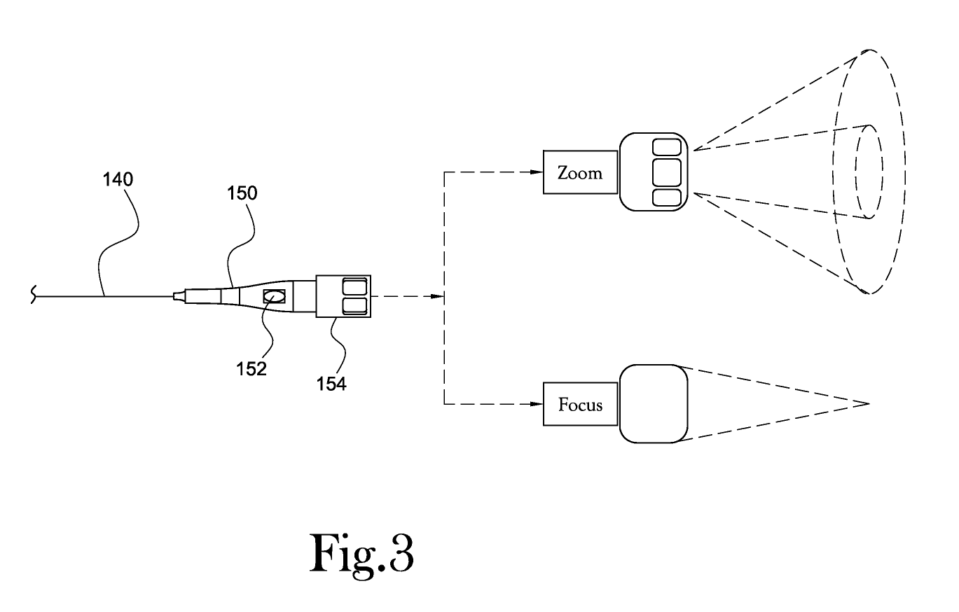

FIG. 3 illustrates example elements and functions of the handpiece 150 of the portable laser emitting apparatus 100 according to an example of the present general inventive concept. As previously described, the handpiece 150 is connected to the portable laser emitting apparatus 100 by the flexible waveguide 140. As also previously described, the handpiece 150 may be provided with at least one switch 152 to enable a user to switch between various laser application modes, and/or to start and stop emission of laser light from the handpiece 150. For example, the at least one switch 152 may be used to switch the handpiece 150 between an ON and OFF state. The at least one switch 152 may also be used to switch between focus and zoom modes of delivery of the laser light. The example handpiece 150 may also be provided with a twist control 154 to enable a user to adjust the focus area of the laser application in a zoom mode.

As illustrated in FIG. 3, the focus mode may be used to concentrate the laser light in a much smaller area of the patient, while the zoom mode may be used to spread the laser light to a comparatively larger area. In one example embodiment, the total area to which the laser light may be applied, according to the distance from the handpiece 150 to the target area, may be approximately 1.0 to 5.0 cm². The focus and zoom modes may be enable by detachable attachments to the handpiece 150, and may be mechanically or magnetically attached to the handpiece 150.

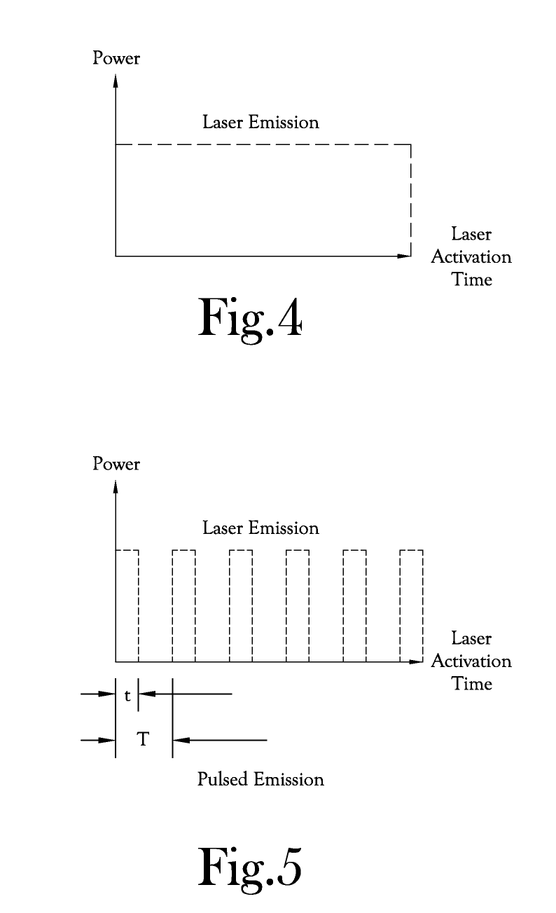

The laser emission may be applied through the handpiece 150 to the patient in several modes according to various example embodiments of the present general inventive concept. For example, according to one example embodiment, the laser light may be applied to the patient in a continuous wave mode, a frequency modulated mode, or an intense super pulse mode. These three example modes are illustrated in FIGS. 4-6.

FIG. 4 is a graph illustrating the application of the laser light in the continuous wave mode. In the continuous wave mode, the laser emission time overlaps the laser activation time, and the power of the laser emission is approximately constant.

FIG. 5 is a graph illustrating the application of the laser light in the frequency modulated, or pulsed emission, mode. In the pulsed emission mode, the laser light is only emitted in periodic pulses during the laser activation time. The laser light is emitted for a time t during each period T of the laser activation time. The duty cycle is defined as the ratio t/T, or:

Duty Cycle=t/T×100%.

The frequency of pulsed emission is the reciprocal of “T”, or:

Frequency of pulsed laser emission (Hz)=1/T.

For example, if the pulsed laser period is 1 millisecond, the frequency of pulsed laser emission would be 1 kHz.

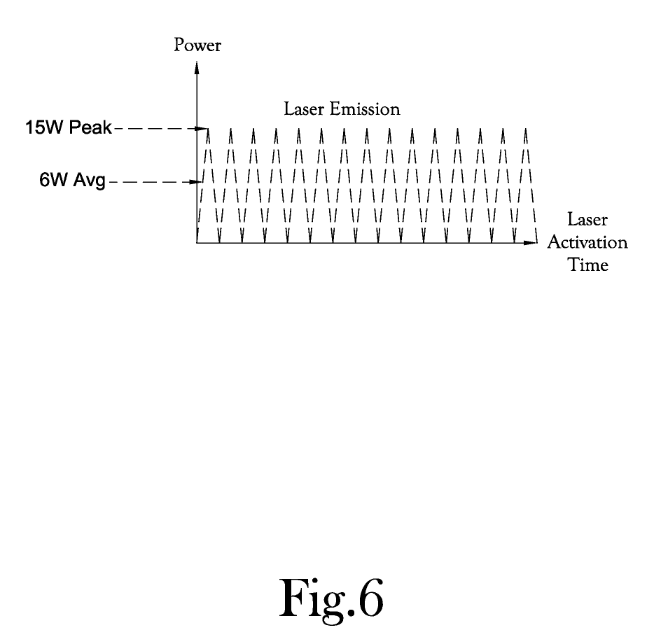

FIG. 6 is a graph illustrating the application of the laser light in the intense super pulse mode. In contrast to the continuous wave and pulsed emission modes, in which the power of the laser light is relatively constant during emission, in the intense super pulse mode the power of the laser emission is periodically and linearly adjusted between a low power and a peak power. In the example intense super pulse mode illustrated in FIG. 6, the peak power of the super pulse is approximately 15 W, while the average power output over time is approximately 6 W.

As previously discussed in regard to FIG. 2, one or more laser sources 220, having the same or different wavelengths, may be provided to the portable laser emitting apparatus 100. The one or more laser sources 220 are provided with current by the respective multi-level current sources 221 to respectively adjust the duration, intensity, and power output of the one or more laser sources 220. A few example embodiments of these one or more laser sources 220, configured and operated accordingly to better treat various medical conditions, will now be described.

In one example embodiment of the present general inventive concept, the portable laser emitting apparatus is provided with a first laser source 220-1 and a second laser source 220-2. The first laser source 220-1 may have a wavelength of 660 nm, with a power output of up to 100 mW, which is known to have stimulatory effects in superficial dermatological conditions such as open wounds, diabetic ulcers and infections. The second laser source 220-2 may have a wavelength of 800 nm, with a power output ranging from 0.1 to 8 or 12 Watts. This second laser source 220-2 is in the NIR range and is centered at the peak of cytochrome c oxidase's absorption.

Another example embodiment may include the first and second laser sources 220-1,220-2 described above, along with a third laser source 220-3, which may have a wavelength of 970 nm, with a power output ranging from 0.1 to 8 or 12 Watts. This wavelength is also in the NIR range and is centered at the peak of water's absorption. This third laser source 220-3 may create thermal gradients on the cellular level along which blood would more readily flow.

Yet another example embodiment may include the first through third laser sources 220-1,220-2,220-3 described above, along with a fourth laser source 220-4, which may have a wavelength of 905 nm, with a power output ranging from 0.1 to 8 or 12 Watts. This fourth laser source 220-4 sits at the peak of oxy-hemoglobin's absorption, thereby increasing the flow of oxygen from the blood to the cells for processing. It should be apparent to one skilled in the art that these are only some of the examples of useful laser wavelengths, and various other embodiments of the present general inventive concept may have any number of other combinations of laser sources, wavelengths, and/or powers. In other words, the present general inventive concept is not limited to the several examples described above.

According to various example embodiments, normal frequency modulated operation, such as that illustrated in FIG. 5, may provide a frequency range adjustable from 1 to 20,000 Hz with an average power ranging from 0.1 to 6 Watts. In various example embodiments of the intense super pulse mode, the frequency range may remain adjustable from 1 to 20,000 Hz, but the peak power may be 21 Watts, with the average power being adjustable from 0.1 to 8 Watts.

The example embodiment illustrated in FIG. 2 also includes various safety features. One such feature is the luminous emission feedback system 280. Included in this luminous emission feedback system 280 is a photodiode 282 and a special direct and reflection optical power system filter 284. Through this filter 284, the photodiode 282 can detect the luminous emissions coming from the irradiated tissues of the patient, which can occur at different wavelengths. These luminous emissions are proportional to the absorption and temperature increase in the patient caused by the emission of laser light from the handpiece 150. The data from the luminous emission feedback system 280 is transmitted to the CPU 210 for analysis and system control. In particular, the luminous emission feedback system 280 insures the efficacy of the optical treatment and maintains the safety level high and constant. Further, this luminous emission feedback system 280 allows an automatic check on the intensity of the laser light delivered to the tissue to optimize the therapeutic effects while guaranteeing maximum safety. Still further, this luminous emission feedback system 280 can gather information for eventual diagnostic purposes.

Another example safety feature in the example embodiment illustrated in FIG. 2 is the use of a cooling system 290 for the one or more laser sources 220. For example, a peltier cooler 292 and fan 294 may be provided to the one or more laser sources 220. Both the peltier cooler 292 and fan 294 may be electrically connected to and under the control of the CPU 210. A temperature gage 296 may be mounted on the one or more laser sources 220 and connected to the CPU 210 so that the CPU 210 can monitor and control the temperature of the one or more laser sources 220 through the control of the peltier cooler 292 and fan 294. This is merely one example through which a safe temperature of the one or more laser sources 220 may be maintained, and is not intended to limit the ways that one can control and maintain proper temperature of the laser sources. One skilled in the art would be aware of other methods for controlling the laser sources in a safe operating temperature range.

A further safety feature illustrated in the example of FIG. 2 is through the providing of a heat sink 296 and a fan 298 on the optical coupler 240, which ensures that the optical coupler 240 remains at a safe operating temperature.

According to various example embodiments of the present general inventive concept, a number of analysis and/or control operations such as, for example, treatment tracking, revenue estimation, credit-based activation, and the like may be available to the user of the portable laser emitting apparatus 100 through interaction with a remotely located device management system. The remote device management system may be a supplier of the portable laser emitting apparatus 100, and/or may track usage information and control various operations of the apparatus 100 as described herein. It is understood that “remote device management system” is simply a term to describe the remotely located managing entity of these services, and is typically an automated management process performed by a processor and memory configuration such as one or more networked server computers, or servers, or similar information processing device, e.g., computer, that is able to communicate data to and from the apparatus 100 through, for example, an internet or cellular connection. In various example embodiments herein, the remote device management system may be referred to as a computer system to manage a remote laser emitting apparatus used in medical treatments.

In various example embodiments, a graphical user interface (GUI) may be displayed on the touch screen display 130 to allow a user of the laser emitting apparatus 100 to track treatments that have been performed and/or will be performed in the future, to estimate current and future revenue based on past and projected future treatments, and/or to perform various other operations that may also be displayed on other electronic communication devices communicating with the remote device management system, such as a smartphone, laptop computer, and the like. The tracked information may be shared with the remote device management system, and the revenue estimates may be provided by the remote device management system, by online or other similar communications between the devices. In various example embodiments, the portable laser emitting apparatus 100 may be authorized to be operated on a credit-based system, wherein the user purchases, through the GUI, a certain number of procedures or time of operation of the device from a remote control location. In various example embodiments, the GUI may be displayed on the touch screen display 130, or may be accessed on any other processing device having a display, such as a computer or smartphone. When displayed on a device other than portable laser emitting apparatus 100, the GUI may be controlled by the CPU 210 of the portable laser emitting apparatus 100 through the communications terminal 270. In other various example embodiments, the GUI may be controlled by, or include information submitted from, other locations, such as a remote site for controlling access to the laser emitting apparatus 100. In other words, various examples of displays describe herein may be displayed on, for example, a laptop computer that is in communication with the remote device management system which is providing the display information. In such an example embodiment, the laptop computer may also be communicating with the portable laser emitting apparatus 100 at the same time, or the portable laser emitting apparatus 100 may receive updated information from the remote device management system or the laptop computer (or other information processing device) at a later time. It is understood that the terms “display” and “GUI” may be used interchangeably in the descriptions of these various example embodiments. The CPU 210 of the laser emitting apparatus 100 may communicate with the remote device management system at the remote site through a variety of ways, such as, for example, a wired internet connection, a wireless connection to a home network, a cellular based connection, and so on.

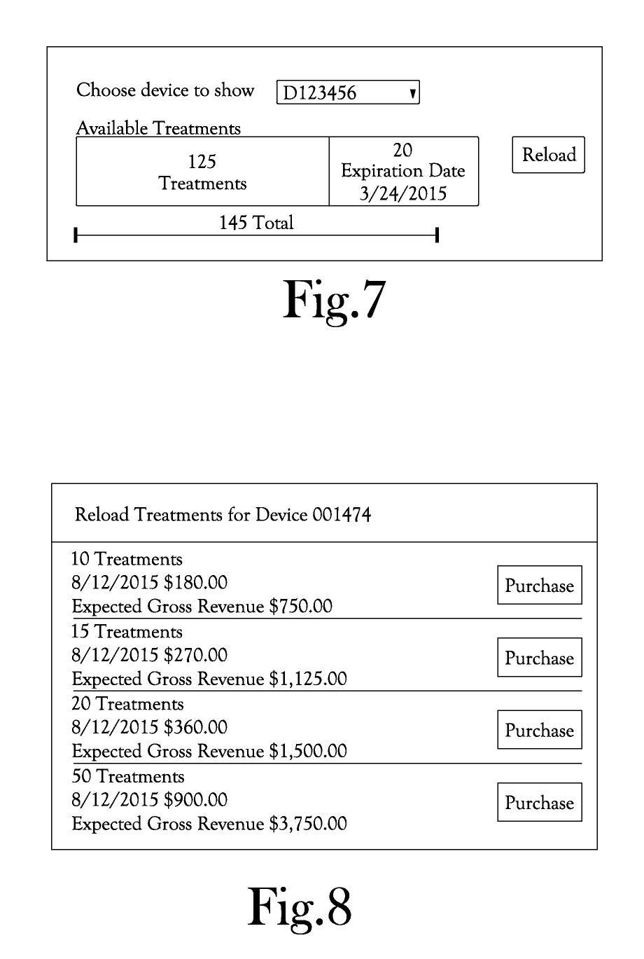

According to various example embodiments of the present general inventive concept, the remote device management system may control a display, to be displayed on a user's device, that presents several categories of information that may be viewed by the user, and which may be selected to display further information according to the selected category. The collection of display information may be referred to herein as a “dashboard” display, and may be the initial display shown to a user upon the user logging in to the remoted device management system's user interface. FIGS. 7-14 illustrate a few example categories/displays that may be displayed in the dashboard display. The dashboard features described herein for access by the user of the laser emitting apparatus may be referred to as user dashboard features, which may be part of the user dashboard, to differentiate from dashboards and/or dashboard features typically accessed by remote device management system administrators.

FIG. 7 illustrates an available treatments display used to track and control operations of the portable laser emitting apparatus according to an example embodiment of the present general inventive concept. The available treatments display illustrated in FIG. 7 may be included in the initial display shown when a user accesses either the laser emitting apparatus 100 itself, or a web site operated by the remote device management system at a remote location controlling credit-based operations of the laser emitting apparatus 100. In FIGS. 7-20, the illustrated displays or portions of displays are shown as being accessed through a web page provided by the remote device management system, such as would be displayed on a laptop computer or the like, but it is understood that the same or similar information may be displayed on the touch screen display 130 of the portable laser emitting apparatus 100, and may be provided to the apparatus 100 by the remote device management system. The system for purchasing credits from the remote device management system at the remote location may be referred to as an on demand system, and such language may be used to refer to the system in the description of various example embodiments described herein. The remotely located entity that controls the credit-based operations and other various assorted data management operations may be referred to in descriptions herein as the remote device management system of the portable laser emitting apparatus operations, or simply the remote device management system.

In various example embodiments, a user may generate, or be provided with, a username and password that are associated with the user's account with the remote device management system. For example, the username and password may be requested by, and provided through, an email exchange with the remote device management system. The user may then access the remote device management system's web page and log into the user's account with the username and password. In various example embodiments, the user may log in and access information from the remote device management system using the touch screen display 130 of the laser emitting apparatus 100.

In various example embodiments, a dashboard display may be displayed that is concentrated in a plurality sections selectable by a user such as, for example, available treatments, expected revenue, history, statistics, etc. The layout of the dashboard display will be described in more detail later in the description of FIG. 14. These sections may be selected by the user upon logging in to access the user's account. For example, the available treatments display illustrated in FIG. 7 allows the user to choose, from a dropdown menu, any devices associated with the user, in the event that more than one device is registered to the user. In the case in which the user is only associated with one device, e.g., one laser emitting apparatus 100, the identification information of that device may simply be displayed without the dropdown menu option. In FIG. 7, the available treatments display shows that the currently displayed device associated with the identification code D123456 has 145 total available treatments, and that 20 of the 145 available treatments are going to expire by Sep. 24, 2015. Therefore, the user has previously made arrangements with the remote device management system to purchase at least 145 treatments, some of which have an expiration date by which they must be used. For example, in some example embodiments the remote device management system assigns purchased credits a 30 day window in which the credits should be used, or the credits may expire at the end of the 30^{th} day. The remaining 125 available treatments indicated in FIG. 7 may also have expiration dates associated therewith, but are not as close to expiring as the 20 treatments for which the expiration date is shown. A reload button may be provided in the available treatments display illustrated in FIG. 7 for the user to purchase additional treatments. In various example embodiments, the different areas in the bar graph indicating the available treatments may be color coded to differentiate between treatments that are and are not in danger of expiring. For example, the treatments that are not close to expiration may be displayed in green, while the treatments that are nearing expiration may be displayed in red. In various example embodiments, if a user clicks the reload button to add additional treatments to the user's account, a display may be triggered to show the user different predetermined purchase options from which the user may choose.

FIG. 8 illustrates a purchase options display used to allow a user to choose from a plurality of purchase options according to an example embodiment of the present general inventive concept. In various example embodiments, if a user selects the reload button illustrated in FIG. 7, the display in FIG. 8 may be displayed to allow the user different predetermined credit package options from which to choose. The purchase options display may be, for example, a pop-up window that is only displayed when the user selects the reload button. As indicated by the example embodiment illustrated in FIG. 8, the user may choose to purchase the treatment credits in quantities of 10, 15, 20, and 50. In various example embodiments, the purchase options display may include incentive information indicating free credits that may be earned by purchasing treatment credit packages of higher quantities. In some example embodiments, the user may be provided with an entry window to enter a different quantity than those shown in the predetermined packages.

In various example embodiments, the newly purchased treatments may be added to the displayed information the next time the user synchronizes the laser emitting apparatus 100 with the remote device management system. In some example embodiments, the laser emitting apparatus 100 may automatically attempt to synchronize with the remote device management system upon the passage of a predetermined time increment. In other example embodiments, the newly purchased treatments may be instantaneously added to the apparatus 100 by the remote device management system communicating with the communications terminal 270 of the apparatus 100, and authorized with the apparatus 100 itself upon the next synchronization or other such communication between the remote device management system and the laser emitting apparatus 100.

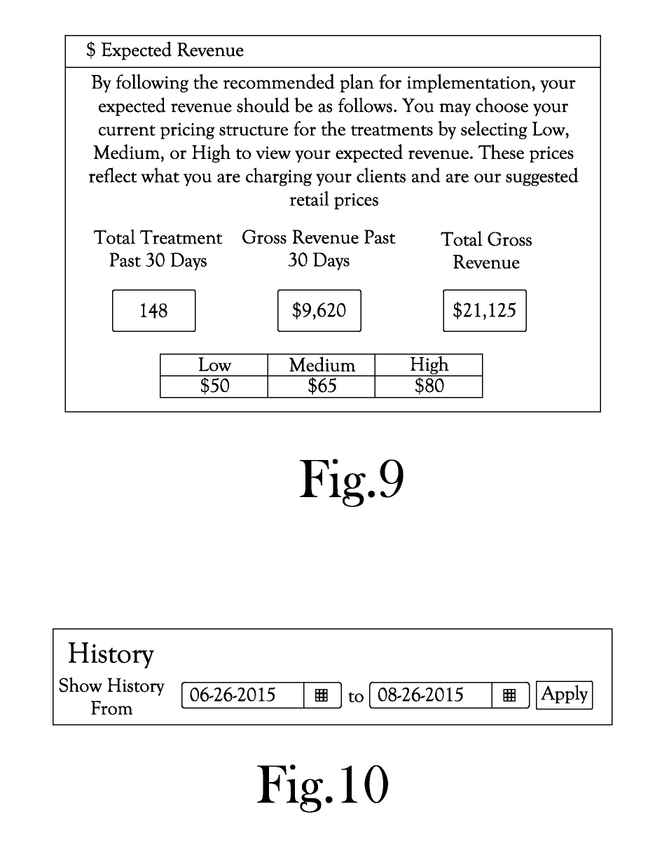

FIG. 9 illustrates an expected revenue display to track and control operations of the portable laser emitting apparatus according to an example embodiment of the present general inventive concept. As illustrated in FIG. 9, the total number of treatments performed for the current 30 day cycle may be displayed along with the gross revenue from the associated cycle. The total gross revenue for the life of the device may also displayed. In various example embodiments, the expected revenue display may advise the user that “By following the recommended plan for implementation, your expected revenue should be as follows. You may choose your current pricing structure for the treatments by selecting Low, Medium, or High to view your expected revenue. These prices reflect what you are charging your clients and are our suggested retail prices.” The Low, Medium, and High estimation of charges may be based on a national average of, for the purpose of this example, $75 a treatment. The number of total treatments performed during a current cycle may be reset every 30 days. The Low, Medium, and High estimations in FIG. 9 may be selectable by the user to show the corresponding revenue totals so that a user may make a more informed decision when determining what price to charge for treatments. In the example illustrated in FIG. 9, the gross revenue for the 148 treatments is based on the selected Medium price of $65. The user may select from the Low and High buttons on the display to see the corresponding differences in those charges per 148 treatments.

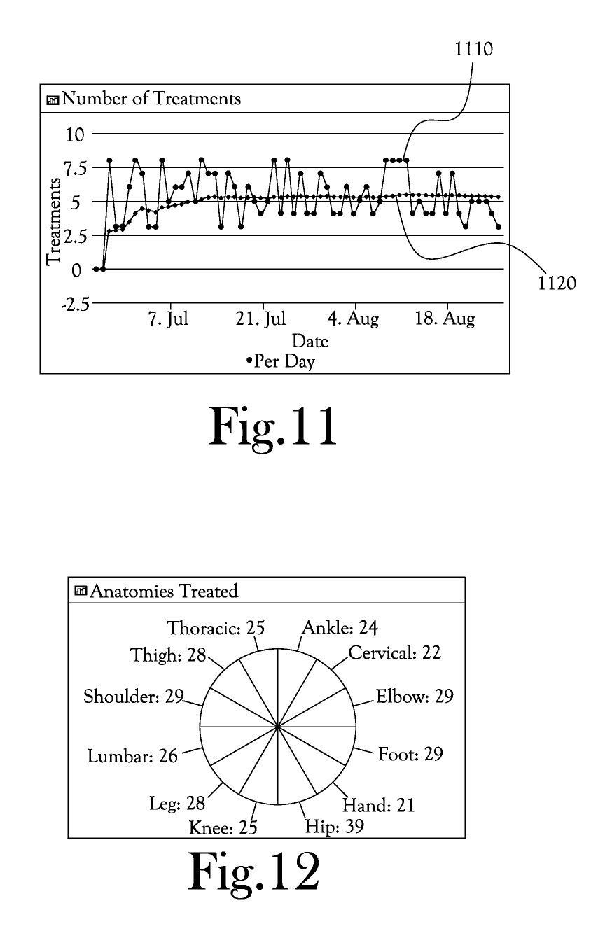

FIG. 10 illustrates a history display used to track and control operations of the portable laser emitting apparatus according to an example embodiment of the present general inventive concept. As illustrated in FIG. 10, the user may be able to select dates, either by, for example, direct alphanumeric entry or through dropdown calendar menus, that define a range for which the user wishes to see associated device information. For example, the associated device information may include a number of treatments performed with the laser emitting apparatus 100, and/or the anatomical regions (or anatomies) treated, during the selected range. FIG. 11 illustrates number of treatments information that may be displayed and associated with the history display of FIG. 10. As illustrated in FIG. 11, the information may be plotted as a number of treatments on the vertical axis, and the date range that was entered by the user on the horizontal axis. The line graph 1110 indicates the total number of treatments per day of the selected time range, and the line graph 1120 indicates the average number of treatments per day over 30-day cycles. In various example embodiments, the user can move a cursor over particular dots of the line graphs to see the exact numbers associated with the selected dots. FIG. 12 illustrates anatomies treated information that may be displayed and associated with the history display of FIG. 10. As illustrated in FIG. 12, the information may be shown in a pie graph, wherein each piece of the pie graph shows the number of treatments performed on an associated portion of the anatomy. The pie chart illustrates a breakdown of anatomies treated by the user during the specified time frame. In various example embodiments, the user can move a cursor over each piece of the pie chart to view the exact number of times the user has treated that particular anatomy. In various example embodiments, the user will enter such anatomy information periodically, such as when a treatment is performed, and the data will be collected and organized by the remote device management system so that the user can access an easy visual representation of the data at his/her leisure. In some example embodiments, the user may enter the anatomical information for desired treatments when purchasing treatment credits, so that the remote device management system may assign treatment time ranges to the treatment credits such that more specific treatment times of use are set according to the desired treatments. For example, treatments for hip or lumbar regions may require longer time than those for fingers or toes, and thus the treatment time per credit can be adjusted accordingly by the remote device management system.

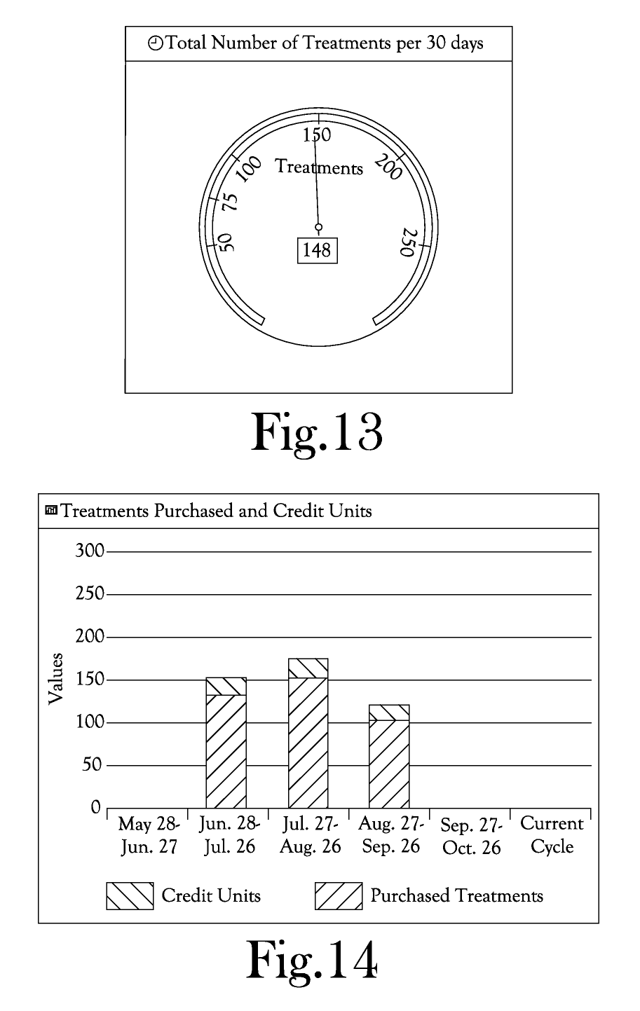

FIG. 13 illustrates total number of treatments information that may be displayed and associated with the history display of FIG. 10. In the example embodiment of FIG. 13, the display is based on a current 30-day cycle of the user, which is indicated as a number on a dial, which may be referred to as a speedometer, with the actual number displayed in the center of the dial. In example embodiments, the display in FIG. 13 is relative to the number of treatment credits applicable to the current 30-day cycle, i.e., the period of time at the end of which one or more of the treatment credits may expire. FIG. 14 illustrates treatments purchased and credit units information that may be displayed and associated with the history display of FIG. 10. In the example embodiment of FIG. 14, the display is a bar chart indicating treatments purchased and credit units. The terms “credit units” and “treatment credits” may be used interchangeably herein. As the user loads the portable laser emitting apparatus 100 with purchased treatment credits, the purchased credits may show up on the bar chart of FIG. 14 with a color or shade indicated below the chart. These purchased treatment credits may be located in the bottom portion of each respective bar of the bar chart. The treatment units that are otherwise credited to the user's account by the remote device management system as, for example, incentive rewards, may be shown on the bar chart with a different color or shade, as also indicated below the chart, and which may be located in the top portion of the respective bars of the bar chart. These credited treatment units may be earned, for example, by purchasing a specific amount of treatment credits during a 30-day cycle or during the initial pre-load of the device. In other words, the purchased treatments indicated in FIG. 14 were actually purchased by the user, while the credit units were credited to the user's account through, for example, an incentive rewards program. In various example embodiments, a user's continued participation in the on demand program may be dependent on the number of credits the user purchases during a 30-day cycle. For example, the user may be provided the laser emitting apparatus for a reduced charge, or no charge, if the user commits to purchasing a certain threshold number of treatment credits every 30 days. Failure to meet these threshold requirements could result in the user having to forfeit the apparatus, or pay a penalty, and so on. These GUI tools aid the user, as well as the remote device management system, in keeping track of such purchases.

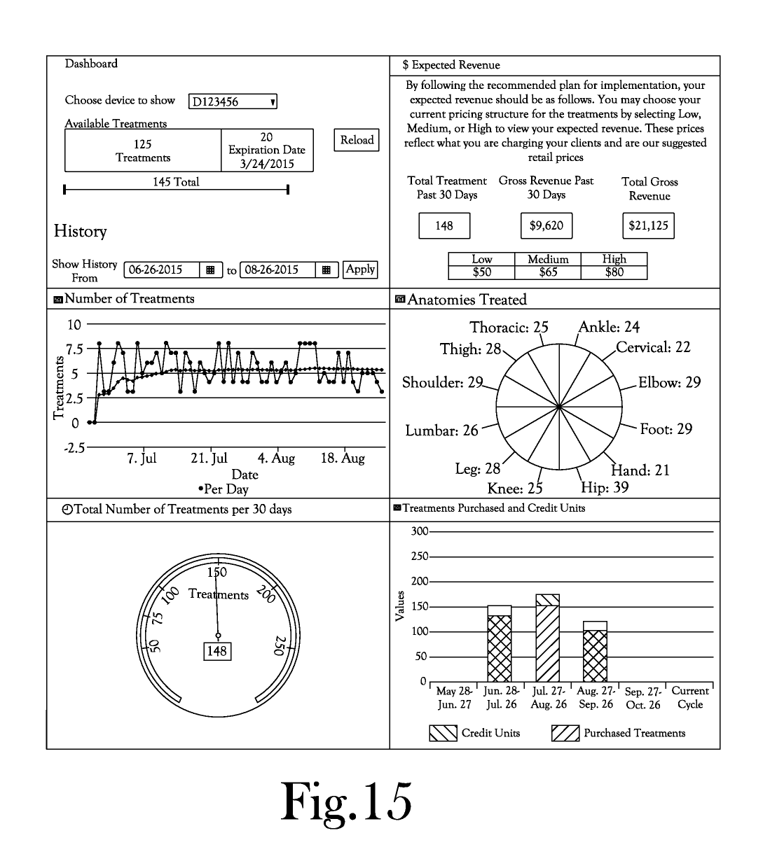

FIG. 15 illustrates a dashboard display including the displays illustrated in FIGS. 7 and 9-14 according to an example embodiment of the present general inventive concept. In this example embodiment, the illustrated dashboard includes all of the previously described displays made available to the user, absent the pop-up menu that results from the user choosing the reload button. It is understood that different example embodiments may have different areas of information, different configurations, and so on, for the user to choose from. Also, the dashboard display may be available in a mobile version for users to access from a device such as a smartphone. In the mobile version of the dashboard display, one or more areas of the display may be represented as a clickable option on the display or a drop-down menu, and the corresponding information may be displayed on a different “page” when the user selects that option.

In various example embodiments of the present general inventive concept, the user may be provided with a user profile through which the remote device management system may securely store user information associated with a corresponding device, payment, and so on. The user information may include general personal information such as name, address, state, phone number, etc., along with the device identification information corresponding to the user information. For example, the user information may include the user's billing information corresponding with a serial number of the corresponding laser emitting apparatus 100. The user may be responsible for keeping such billing information up to date and making any necessary changes to ensure uninterrupted on demand service from the remote device management system. The user account may be initiated by the user sending user information to the remote device management system, for example, by email, instant messaging, and/or interacting with the remote device management system interface at a web page administered by the remote device management system, at which point the user profile may be set up in the system. Once the user confirms the user account, in various example embodiments the user may easily view and edit information through remote device management system interface, which may be referred to as the on demand dashboard, at any time.

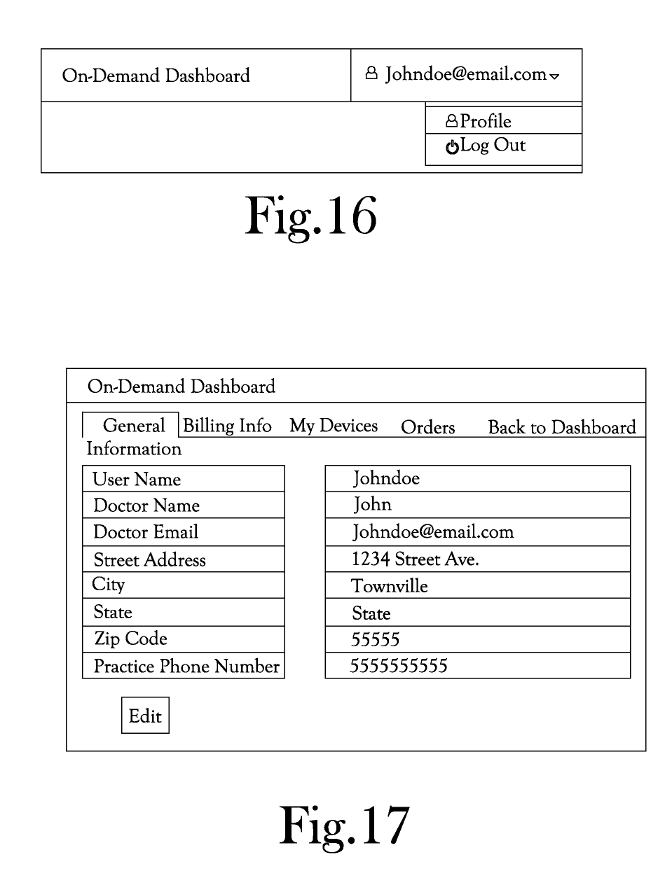

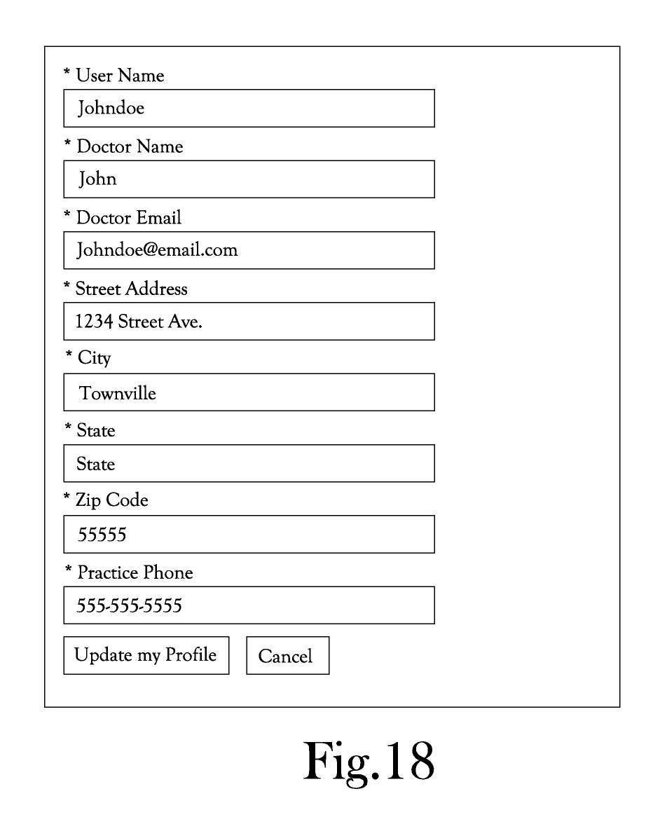

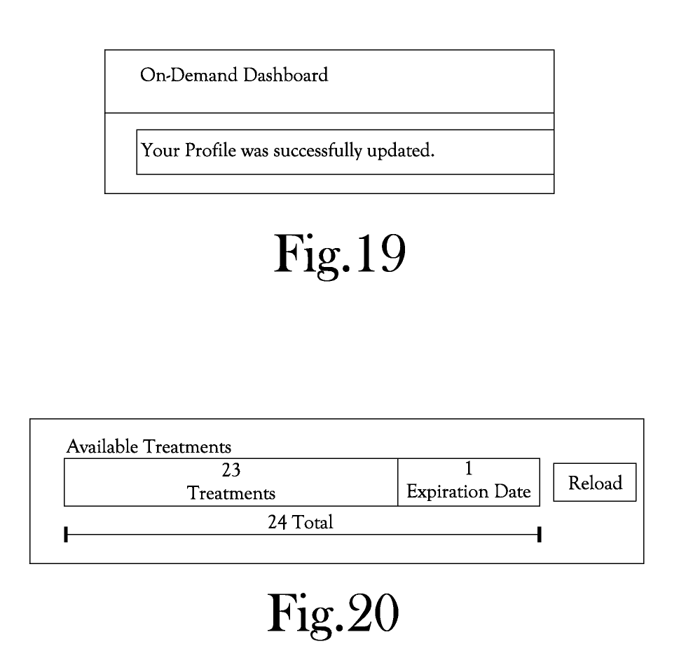

In various example embodiments, the user can update the user information by going to an on demand web site of the remote device management system and log in with the user's username and password that has been previously established. FIG. 16 illustrates a logged in display through which a user may update a user profile according to an example embodiment of the present general inventive concept. The display of FIG. 16 indicates that the user is logged in by showing the user's identification (an email address in this example) and a log out option. In this example embodiment, the user simply clicks on “Profile” to bring up profile information to be edited or verified. FIG. 17 illustrates a user profile display according to an example embodiment of the present general inventive concept. In the example embodiment shown in FIG. 17, the user may simply click the tab of the type of information that the user wishes to edit, such as general information, billing information, devices, orders, etc. One or more fields may then be displayed according to the chosen tab. In the example embodiment of FIG. 17, the user has chosen the General Information tab, and corresponding fields for user name, doctor name, doctor email, and practice phone number are displayed. The user may click the Edit button to edit one or more of the displayed fields. FIG. 18 illustrates an updatable information display for the user to edit user information according to an example embodiment of the present general inventive concept. In the example embodiment of FIG. 18, the user can simple change any of the information displayed in the text fields to update the corresponding information, and then select the Update My Profile button to save the changed data. FIG. 19 illustrates a successful update notification display according to an example embodiment of the present general inventive concept. In the example embodiment of FIG. 19, the user is notified by text that the user profile has been successfully updated. In various example embodiments, the text notifying the user of the successful update may be located in a color-coded box having, for example, a green background, to further indicate that update was successful.

In various example embodiments of the present general inventive concept, as previously discussed, a user may add treatment credits to the corresponding laser emitting apparatus 100 through the on demand website discussed above, and/or directly through the laser emitting apparatus 100. FIG. 20 illustrates another example of the available treatment display of FIG. 7 according to an example embodiment of the present general inventive concept. In order to purchase treatment credits through the on demand portal provided by the remote device management system, after logging in the user may simply choose the available treatments display. In various example embodiments, the available treatments display may be included in the initial on demand dashboard display, or may be accessed by the user by clicking an available treatments button. As previously described, the user may then click the reload button, which may be color-coded to aid in quick identification by the user, next to the bar chart displaying the number of available treatments. In some example embodiments, a pop-up display such as that illustrated in FIG. 8 may be displayed to allow the user to select from various predetermined packages of treatment credits. According to various example embodiments, upon selecting the reload button the user may be prompted to simply enter the number of treatments the user would like to purchase. In various example embodiments, the treatments may be available in packages of 5, 10, 20, 50, and so on. Certain quantities may qualify the user for free credit units as discussed previously in regard to FIG. 14. After choosing the desired quantity, the user may execute the transaction, for example, by clicking a purchase button which authorizes the remote device management system to bill the user accordingly. In various example embodiments, the purchase may be further confirmed by another confirmation button that the user must click after clicking the purchase button. In various example embodiments, the treatment credits may be added to the user's account and visible in the remote device management system's on demand dashboard instantly, and purchased credits may appear and be available for use on the user's laser emitting apparatus 100 after the apparatus 100 has communicated with the remote device management system, for example, by a synchronization over a Wi-Fi or other such connection. The user may also be provided with a confirmation email, text message, etc., that includes the purchase details corresponding to the transaction.

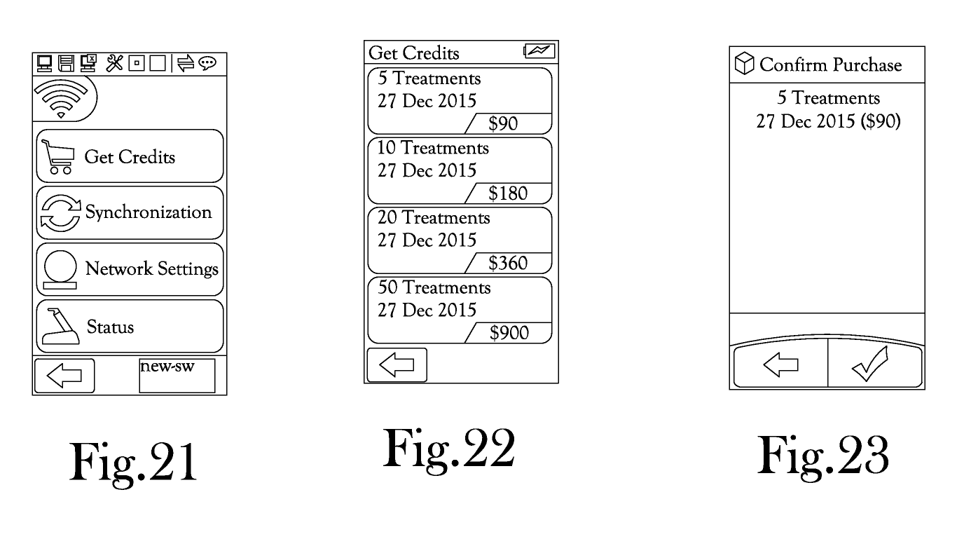

In various example embodiments of the present general inventive concept, the user may be able to purchase treatment credits from the remote device management system directly through the portable laser emitting apparatus 100, which may also be referred to as an on demand device. In various example embodiments, the apparatus 100 may need to be in communication with the remote device management system during the purchase, such as through a wired or wireless internet connection, a cellular connection, or the like. FIGS. 21-23 illustrate example interactive displays used to purchase treatment credits through the laser emitting apparatus according to an example embodiment of the present general inventive concept. Upon turning the laser emitting apparatus 100 on using the on/off button, the user may be prompted to enter a PIN code or other such identification information that may be used to unlock various processes of the apparatus 100. In example embodiments in which the entry of the PIN code is used, upon entry the home screen of the apparatus 100 may be displayed on the touch-screen display 130. FIG. 21 illustrates an example embodiment of the home screen of the apparatus 100. From the home screen of FIG. 21, the user may select several operations such as, for example, Get Credits, Synchronization, Network Settings, Status, and so on. To purchase treatment credits, the user may select the Get Credits button. In various example embodiments, the touch-screen display 130 will display one or more available treatment packages, as illustrated in FIG. 22. The treatment credit packages illustrated in FIG. 22 include quantities of 5, 10, 20, and 50, along with the corresponding prices and expiration dates for each package. The user may select the treatment package that the user desires to purchase by selecting the corresponding button on the touch-screen display 130. According to various example embodiments, the user may then be presented with the display shown in FIG. 23, which displays the treatment credit package selected by the user, along with the corresponding price and expiration date, and the user is able to confirm the purchase by selecting the appropriate button on the screen. The apparatus will then communicate with the remotely located remote device management system, either immediately because of the purchase by the user, or at a later time when either the user synchronizes the device with the remote device management system or when the remote device management system initiates a communication, so that the device will be authorized to use the newly purchased treatment credits. According to various example embodiments, the remote device management system may send an email, text message, or the like to the user to memorialize the purchase details. Such details may also be displayed by the apparatus 100 following a communication with, and an authorization by, the remote device management system.

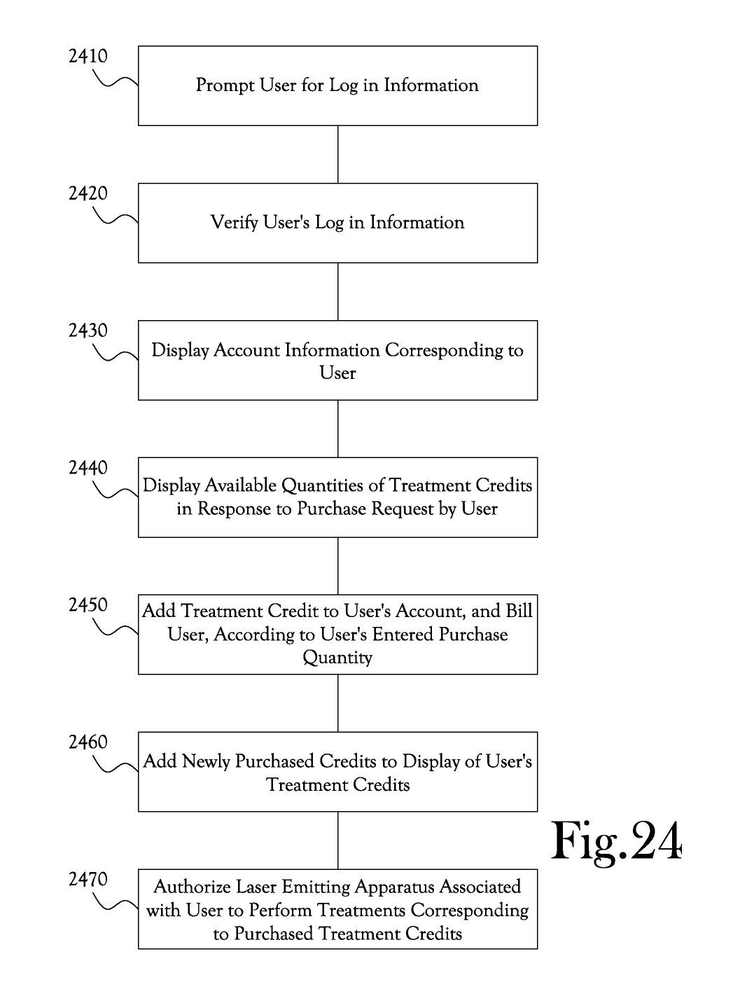

FIG. 24 illustrates a method of controlling the portable laser emitting apparatus according to an example embodiment of the present general inventive concept. It is understood that no specific order is implied for all of the operations illustrated in FIG. 24, and various other example embodiments may include more or fewer operations. Additionally, the example embodiment illustrated in FIG. 24 may include operations not explicitly listed in FIG. 24, and/or sub-operations that correspond to the listed operations. When an interface such as the previously discussed on demand dashboard is accessed by a user, e.g., when a computer system to manage a remote laser emitting apparatus used in medical treatments is contacted by the user through, for example, a web page, the computer system, which may be otherwise referred to as the remote device management system, may prompt the user for log in information in operation 2410. When the user's log in information has been entered, the remote device management system may verify the log in information by comparing the entered log information with information previously stored in one or more data storages of the remote device management system in operation 2420. Once the user's log in information is verified, the remote device management system may display the user's account information in operation 2430, through which the user can edit information, review treatment history, etc., as well as purchase treatment credits. In the event that the user indicates a desire to purchase treatment credits, the remote device management system may display available quantities of treatment credits to the user in operation 2440. In operation 2450, the remote device management system may add the treatment credits to the user's account, and bill the user, according to the desired purchase quantity entered by the user. The remote device management system may add the newly purchased treatment credits to the display of the user's total number of treatment credits in operation 2460. In operation 2470, the remote device management system may authorize the portable laser emitting apparatus associated with the user through the user's account information to perform the number of treatments corresponding to the number of purchased treatment credits. The authorized number of treatments may include any previously purchased treatment credits with the newly purchased treatment credits, according to whether any of the treatment credits have expired due to use and/or expiration dates. According to various example embodiments, all of the purchased and otherwise obtained treatment credits may have a corresponding expiration date associated therewith.

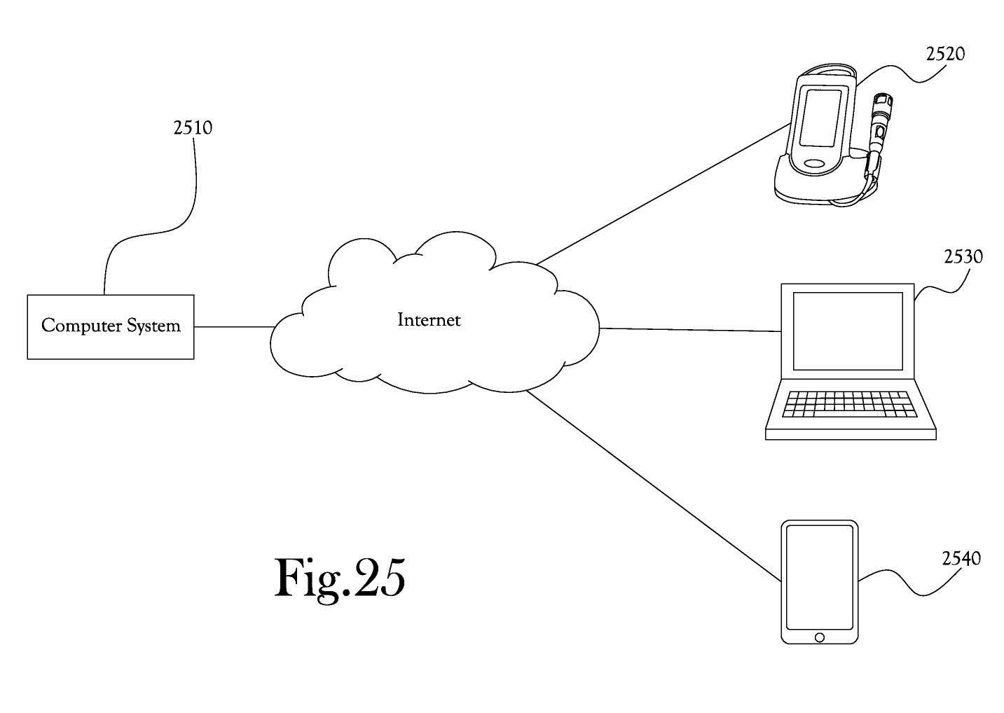

FIG. 25 illustrates a system to perform laser therapy including a computer system in communication with a portable laser emitting apparatus, laptop computer, and/or smartphone according to an example embodiment of the present general inventive concept. It is understood that any of a number of electronic communication devices with displays may be used to communicate with the computer system managing the laser emitting apparatus, and that the system to perform laser therapy is not limited to those items illustrated in FIG. 25. In the example embodiment illustrated in FIG. 25, the computer system 2510 may communicate with the laser emitting apparatus 2520 through a communication path such as the internet, which may also be used to communicate with the laptop computer 2530 and the smartphone 2540. As previously described, in other various example embodiments the computer system 2510 may communicate with the illustrated devices through a cellular communication connection. In various example embodiments, the laser emitting apparatus 2520, laptop computer 2530, and/or smartphone 2540 may communicate with one another or the computer system 2510 through a Wi-Fi connection.

According to various example embodiments of the present general inventive concept, a method of using a computer system to manage a remote laser emitting apparatus used in medical treatments may include the computer system receiving, through computer data communication, apparatus identification information associated with a remotely located laser emitting apparatus, and user identification information associated with a user of the laser emitting apparatus, the computer system storing the apparatus identification information and user identification information, the computer system correlating, in response to receiving data regarding a purchase of one or more treatment credits by the user, the purchased treatment credits to the laser emitting apparatus associated with the user, and the computer system communicating the purchased treatment credits to the laser emitting apparatus through computer data communication, wherein the purchased treatment credits authorize the laser emitting apparatus to perform a number of treatments corresponding to the purchased treatment credits. The computer data communication may be communicated via the internet. The data regarding the purchase of the one or more treatment credits by the user may be received from the laser emitting apparatus. The data regarding the purchase of the one or more treatment credits may be entered directly into the laser emitting apparatus by the user. The method may further include the computer system controlling a user interface so as to be displayed on a remote device, wherein the data regarding the purchase of the one or more treatment credits by the user is facilitated through the user interface. The user interface may be a web page displayed on an information processing device. The method may further include the computer system prompting the user for the user identification information at an initial display of the user interface. The method may further include the computer system displaying in the user interface a total number of treatments for which the laser emitting apparatus is currently authorized. The method may further include the computer system displaying in the user interface an expiration date by which one or more of the corresponding authorized treatments must be performed. The method may further include the computer system displaying in the user interface an option for the user to purchase additional treatment credits. The method may further include the computer system displaying in the user interface various components indicating expected revenue based on data collected from the laser emitting apparatus through computer data communication. The method may further include the computer system displaying in the user interface historical data regarding usage of the laser emitting apparatus according to a date range entered by the user through the user interface. The method may further include the computer system communicating with the laser emitting apparatus through computer data communication to compile the historical data. The historical data may include at least one of treatment dates or anatomies treated. The method may further include the computer system displaying in the user interface statistical data regarding laser emitting apparatus usage and/or treatment credits based on one or more monthly cycles.

According to various example embodiments of the present general inventive concept, a portable laser emitting apparatus to be used in physiotherapy and/or surgery may be provided, the apparatus including a readily portable housing, one or more laser sources of same or different wavelengths provided in the housing, a flexible waveguide extending from the housing and configured to transmit laser light from the one or more laser sources to a target area, a data storage configured to store a plurality of predetermined settings of wavelength and power combinations to be emitted from the one or more laser sources, data corresponding to past usage of the laser emitting apparatus, and treatment credit information regarding whether the laser emitting apparatus is authorized to perform one or more treatments, a communication terminal configured to receive data updates for the controller and/or data storage from a remote device management system, and a controller configured to control the one or more laser sources to operate according to the predetermined settings in response to the laser emitting apparatus being authorized to perform a treatment, the past usage data, and communication with the remote device management system to share the usage data and/or receive new treatment credits. The controller may be configured to perform a quantity of treatments that corresponds to a number of treatment credits stored in the data storage. The treatment credits may be communicated to the laser emitting apparatus from the remote device management system. The portable laser emitting apparatus may further include a touch screen display configured to display usage information and to receive treatment credit purchase information from a user.

According to various example embodiments of the present general inventive concept, a non-transitory storage medium may be provided having recorded thereon machine readable instructions to cause a computer system to perform a method of managing a remote laser emitting apparatus used in medical treatments, the method including the computer system receiving, through computer data communication, apparatus identification information associated with a remotely located laser emitting apparatus, and user identification information associated with a user of the laser emitting apparatus, the computer system storing the apparatus identification information and user identification information, the computer system correlating, in response to receiving data regarding a purchase of one or more treatment credits by the user, the purchased treatment credits to the laser emitting apparatus associated with the user, and the computer system communicating the purchased treatment credits to the laser emitting apparatus through computer data communication, wherein the purchased treatment credits authorize the laser emitting apparatus to perform a number of treatments corresponding to the purchased treatment credits.

According to various example embodiments of the present general inventive concept, a system to perform laser therapy may be provided, including a portable laser emitting apparatus configured to perform laser therapy, and a remotely located computer system configured to receive, through computer data communication, apparatus identification information associated with the laser emitting apparatus, and user identification information associated with a user of the laser emitting apparatus, to store the apparatus identification information and user identification information, to correlate, in response to receiving data regarding a purchase of one or more treatment credits by the user, the purchased treatment credits to the laser emitting apparatus associated with the user, and to communicate the purchased treatment credits to the laser emitting apparatus through computer data communication, wherein the purchased treatment credits authorize the laser emitting apparatus to perform a number of treatments corresponding to the purchased treatment credits.

According to various embodiments of the present general inventive concept, a portable laser emitting apparatus is provided which may be readily moved from location to location in a home or office, either in the same room or different rooms. The portable laser emitting apparatus is constructed such that it is lightweight enough to be easily moved by hand, and through a provided waveguide conveniently transmit laser light from one or more laser sources to a patient.

It is noted that the simplified diagrams and drawings do not illustrate all the various connections and assemblies of the various components, however, those skilled in the art will understand how to implement such connections and assemblies, based on the illustrated components, figures, and descriptions provided herein, using sound engineering judgment.

Various example embodiments of the present general inventive concept described herein may include operations performed by one or more processors, computers, etc., that are caused to perform these operations by instructions recorded on a non-transitory computer readable storage medium. Various ones of the operations and processes described and/or associated with the described various operations and processes may be performed on any of a host of devices, such as an online server, personal computer, smart phone, tablet computer, etc., or any device containing one or more processors to process such instructions.

Program instructions to perform a method described herein, or one or more operations thereof, may be recorded, stored, or fixed in one or more non-transitory computer-readable storage media. The program instructions may be implemented by a computer. For example, the computer may cause a processor to execute the program instructions. The media may include, alone or in combination with the program instructions, data files, data structures, and the like. Examples of computer-readable media include magnetic media, such as hard disks, floppy disks, and magnetic tape; optical media such as CD ROM disks and DVDs; magneto-optical media, such as optical disks; and hardware devices that are specially configured to store and perform program instructions, such as read-only memory (ROM), random access memory (RAM), flash memory, and the like. Examples of program instructions include machine code, such as produced by a compiler, and files containing higher level code that may be executed by the computer using an interpreter. The program instructions, that is, software, may be distributed over network coupled computer systems so that the software is stored and executed in a distributed fashion. For example, the software and data may be stored by one or more computer readable recording mediums. Also, functional programs, codes, and code segments for accomplishing the example embodiments disclosed herein can be easily construed by programmers skilled in the art to which the embodiments pertain based on and using the flow diagrams and their corresponding descriptions as provided herein. Also, the described unit to perform an operation or a method may be hardware, software, or some combination of hardware and software. For example, the unit may be a software package running on a computer or the computer on which that software is running.

Numerous variations, modifications, and additional embodiments are possible, and accordingly, all such variations, modifications, and embodiments are to be regarded as being within the spirit and scope of the present general inventive concept. For example, regardless of the content of any portion of this application, unless clearly specified to the contrary, there is no requirement for the inclusion in any claim herein or of any application claiming priority hereto of any particular described or illustrated activity or element, any particular sequence of such activities, or any particular interrelationship of such elements. Moreover, any activity can be repeated, any activity can be performed by multiple entities, and/or any element can be duplicated.

While the present general inventive concept has been illustrated by description of several example embodiments, it is not the intention of the applicant to restrict or in any way limit the scope of the inventive concept to such descriptions and illustrations. Instead, the descriptions, drawings, and claims herein are to be regarded as illustrative in nature, and not as restrictive, and additional embodiments will readily appear to those skilled in the art upon reading the above description and drawings.

1. A method of using a computer system to manage a remote laser emitting apparatus used in medical treatments, comprising:
the computer system receiving, through computer data communication, apparatus identification information associated with a remotely located laser emitting apparatus, and user identification information associated with a user of the laser emitting apparatus;
the computer system storing the apparatus identification information and user identification information;
the computer system correlating, in response to receiving data regarding a purchase of one or more treatment credits by the user, the purchased treatment credits to the laser emitting apparatus associated with the user; and
the computer system communicating the purchased treatment credits to the laser emitting apparatus through computer data communication;
wherein the purchased treatment credits authorize the laser emitting apparatus to perform a number of treatments corresponding to the purchased treatment credits.

2. The method of claim 1, wherein the computer data communication is communicated via the internet.

3. The method of claim 1, wherein the data regarding the purchase of the one or more treatment credits by the user is received from the laser emitting apparatus.

4. The method of claim 3, wherein the data regarding the purchase of the one or more treatment credits is entered directly into the laser emitting apparatus by the user.

5. The method of claim 1, further comprising the computer system controlling a user interface so as to be displayed on a remote device, wherein the data regarding the purchase of the one or more treatment credits by the user is facilitated through the user interface.

6. The method of claim 5, wherein the user interface is a web page displayed on an information processing device.

7. The method of claim 5, further comprising the computer system prompting the user for the user identification information at an initial display of the user interface.

8. The method of claim 5, further comprising the computer system displaying in the user interface a total number of treatments for which the laser emitting apparatus is currently authorized.

9. The method of claim 8, further comprising the computer system displaying in the user interface an expiration date by which one or more of the corresponding authorized treatments must be performed.

10. The method of claim 8, further comprising the computer system displaying in the user interface an option for the user to purchase additional treatment credits.

11. The method of claim 5, further comprising the computer system displaying in the user interface various components indicating expected revenue based on data collected from the laser emitting apparatus through computer data communication.

12. The method of claim 5, further comprising the computer system displaying in the user interface historical data regarding usage of the laser emitting apparatus according to a date range entered by the user through the user interface.

13. The method of claim 12, further comprising the computer system communicating with the laser emitting apparatus through computer data communication to compile the historical data.

14. The method of claim 12, wherein the historical data includes at least one of treatment dates or anatomies treated.

15. The method of claim 5, further comprising the computer system displaying in the user interface statistical data regarding laser emitting apparatus usage and/or treatment credits based on one or more monthly cycles.

16. A portable laser emitting apparatus to be used in physiotherapy and/or surgery, the apparatus comprising:
a readily portable housing;
one or more laser sources of same or different wavelengths provided in the housing;
a flexible waveguide extending from the housing and configured to transmit laser light from the one or more laser sources to a target area;
a data storage configured to store a plurality of predetermined settings of wavelength and power combinations to be emitted from the one or more laser sources, data corresponding to past usage of the laser emitting apparatus, and treatment credit information regarding whether the laser emitting apparatus is authorized to perform one or more treatments;
a communication terminal configured to receive data updates for the controller and/or data storage from a remote device management system; and
a controller configured to control the one or more laser sources to operate according to the predetermined settings in response to the laser emitting apparatus being authorized to perform a treatment, the past usage data, and communication with the remote device management system to share the usage data and/or receive new treatment credits.

17. The portable laser emitting apparatus of claim 16, wherein the controller is configured to perform a quantity of treatments that corresponds to a number of treatment credits stored in the data storage.

18. The portable laser emitting apparatus of claim 17, wherein the treatment credits are communicated to the laser emitting apparatus from the remote device management system.

19. The portable laser emitting apparatus of claim 16, further comprising a touch screen display configured to display usage information and to receive treatment credit purchase information from a user.

20. A non-transitory storage medium having recorded thereon machine readable instructions to cause a computer system to perform a method of managing a remote laser emitting apparatus used in medical treatments, the method comprising:
the computer system receiving, through computer data communication, apparatus identification information associated with a remotely located laser emitting apparatus, and user identification information associated with a user of the laser emitting apparatus;
the computer system storing the apparatus identification information and user identification information;
the computer system correlating, in response to receiving data regarding a purchase of one or more treatment credits by the user, the purchased treatment credits to the laser emitting apparatus associated with the user; and
the computer system communicating the purchased treatment credits to the laser emitting apparatus through computer data communication;
wherein the purchased treatment credits authorize the laser emitting apparatus to perform a number of treatments corresponding to the purchased treatment credits.

21. A system to perform laser therapy, comprising:
a portable laser emitting apparatus configured to perform laser therapy; and
a remotely located computer system configured to receive, through computer data communication, apparatus identification information associated with the laser emitting apparatus, and user identification information associated with a user of the laser emitting apparatus, to store the apparatus identification information and user identification information, to correlate, in response to receiving data regarding a purchase of one or more treatment credits by the user, the purchased treatment credits to the laser emitting apparatus associated with the user, and to communicate the purchased treatment credits to the laser emitting apparatus through computer data communication;
wherein the purchased treatment credits authorize the laser emitting apparatus to perform a number of treatments corresponding to the purchased treatment credits.
